(19) United States
(12) Patent Application Publication
(10) Pub. No.: US 2009/0197815 A1
(43) Pub. Date: Aug. 6, 2009

(54) COMBINATION OF SOMATOSTATIN-ANALOGS WITH DOPAMINE- OR GROWTH HORMONE RECEPTOR ANTAGONIST

(76) Inventors: Beatrice Vincenzi, Verona (IT); Herbert Schmid, Neuenburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/303,502

(22) PCT Filed: Jun. 6, 2007
(86) PCT No.: PCT/EP2007/055599

§ 371 Date: Dec. 4, 2008

Related U.S. Application Data
(60) Provisional application No. 60/804,200, filed on Jun. 8, 2006.

(51) Int. Cl. A61K 38/08; A61P 5/08

(52) U.S. Cl. 514/16

(57) ABSTRACT

The present invention concerns a product containing Sandostatin® LAR® at 40 mg/28 days or pasireotide and either a dopamine-agonist, preferably cabergoline, or a growth hormone receptor antagonist, preferably pegvisomant, as a combined preparation for simultaneous, separate or sequential use in acromegalic therapy. In particular, this therapy is useful for treating acromegalic patients not achieving biochemical normalization after at least six-month treatment using at least one somatostatin analogue at conventional regimen.

The present invention relates to a therapeutic treatment of acromegaly and its complications.

More precisely, the present invention concerns a product containing a long-acting repeatable octreotide acetate, e.g. Sandostatin® LAR®, at 40 mg/28 days or pasireotide and either a dopamine-agonist, preferably cabergoline, or a growth hormone receptor antagonist, preferably pegvisomant, as a combined preparation for simultaneous, separate or sequential use in acromegalic therapy. In particular, the therapy according to the invention is useful for treating acromegalic patients not achieving biochemical normalization after at least six-month treatment using at least one somatostatin analogue at conventional regimen.

Acromegaly is a clinical and metabolic disease caused in more than 95% of patients by growth hormone (GH) hypersecretion from a pituitary adenoma. Acromegaly is an insidious, chronic disease that is associated with bony and soft tissue overgrowth. Most patients experience an increase in hand, foot and head size, broadening of the jaw, enlargement of the tongue and coarsening of the facial features. Many organs, including the liver and kidneys enlarge. Common clinical symptoms include headache, excessive perspiration, fatigue, paresthesiae, weakness, joint pain, and weight gain. Patients may also present with osteoarthritis, carpal tunnel syndrome, visual abnormalities, sleep apnea, or reproductive disorders.

Hypersecretion of GH results in elevated levels of plasma circulating insulin-like growth factor (IGF-I) that is primarily responsible for the majority of the clinical symptoms of acromegaly, and can be elevated even in patients with minimally active disease (Barkan et al., 1997). While the physical presence of the pituitary tumor mass causes some morbidity, the effects of elevated GH and IGF-I levels contribute to a 2-3 fold increase in mortality (Acromegaly Therapy Consensus Development Panel, 1994). Premature death mainly results from cardiovascular, cerebrovascular, respiratory complications or metabolic disturbances such as diabetes mellitus, and a predisposition to gastrointestinal cancer (Colao et al., 2004). Epidemiological data has demonstrated so far that the level of GH secretion is associated with an increased mortality and morbidity rate. Indeed, acromegalic patients attaining GH levels <2.5 μg/L have been shown to approach a survival rate equal to the one of the normal, age-matched population. Causative factors and relationship to IGF-1 are still not clearly detected. The analysis of the West Midland Pituitary Database (Ajuk et al., 2004), covering information of 419 acromegalic patients, demonstrated that reduction of GH levels to <2 μg/L was beneficial in terms of long term outcome and that the sole use of IGF-1 as a marker for effective treatment was not justified.

There is therefore a need for a therapy that would make it possible to (D eradicate the tumor, \textcircled{2} suppress GH secretion to safe values, \textcircled{3} normalize IGF-I levels, \textcircled{4} preserve or restore normal pituitary function, and \textcircled{5} reverse metabolic and clinical abnormalities.

Trans-sphenoidal surgical resection is recommended for most patients with well-localized microadenomas (Melmed et al., 1998) (diameter of 10 mm or less) as this approach has the advantage of producing a rapid therapeutic response. GH concentration may fall to normal within hours and soft tissue enlargement may improve, even before the patient has been discharged from the hospital. Patients with invasively growing macroadenomas (diameter of greater than 10 mm) typically have a poorer prognosis following surgical resection, with surgical cure (defined as GH suppressed to <2.5 μg/L) typically less than 50%, particularly in those with extrasellar extension (Acromegaly Therapy Consensus Development Panel, 1994). Nevertheless as published data are usually generated in centers of excellence, and therefore might not reflect the standard outcome, the overall surgical cure rate should be probably closer to 20-40% when more stringent criteria of <2.5 μg/L and normalization of IGF-I are used (Barkan et al., 1997). Side effects of surgery include local complications (cerebrospinal fluid leak, arachnoiditis), permanent diabetes insipidus, and pituitary failure (Acromegaly Therapy Consensus Development Panel, 1994) and many of those patients who are defined as “cured” will continue to exhibit elevated GH levels when retested one or more years post surgery (Fahlbusch et al., 1994).

Although radiation has been considered second line therapy following surgery, a recent publication has suggested that radiotherapy is ineffective in normalizing IGF-I in acromegalic patients (Barkan et al., 1997). Ajuk et al. found, that treatment with radiotherapy was associated with increased mortality, with cerebrovascular disease predominantly cause of death (Ajuk et al., 2004). Even when radiation is effective, it takes more than two years before a decrease in GH is noted, and up to 20 years for 90% of patients to achieve GH levels of <5.0 μg/L (Acromegaly Therapy Consensus Development Panel, 1994). Irradiation results in hypopituitarism in more than 50% of patients (Acromegaly Therapy Consensus Development Panel, 1994) and may rarely result in visual disturbances, development of secondary brain malignancies, brain necrosis, or brain damage (Jones, 1994).

So far, the medical treatment of choice for acromegalic patients are somatostatin analogues (SSA), that are employed to achieve rapid suppression of GH secretion after incomplete tumor removal either as an adjuvant to radiotherapy, as second line therapy after surgery, or as an alternative to surgery or irradiation in patients who are not candidates for these procedures. Classically-used SSA include, e.g., octreotide, lanreotide, pasireotide and vapreotide (RC-160).

Recent studies have shown that biochemical control (defined as GH<2.5 μg/L and IGF-1 within the age- and sex-adjusted normal range) can be achieved in 40 to 50% of acromegalic patients treated with SSA (Freda et al., 2005).

Sandostatin® LAR® (octreotide acetate) is a long-acting synthetic SSA with a half-life of 80-100 minutes, that was first used to treat acromegaly. Initial studies demonstrated the effectiveness of Sandostatin® in treating patients with acromegaly, with GH levels decreasing to <5.0 μg/L in 65% of patients and to <2.0 μg/L in 40% of patients and normalization of IGF-I in approximately 60% of cases (Newman et al. 1995).

Because of its efficacy and safety profile, Sandostatin®, e.g. Sandostatin® LAR® has become the preferred medical therapy for acromegaly. Sandostatin® LAR® (Long Acting Repeatable) is a one-month sustained release formulation wherein octreotide is incorporated into microspheres of the biodegradable polymer, poly (D,L-lactide-co-glycolide)glucose, as disclosed in U.S. Pat. No. 5,538,739 of Jul. 23, 1996.

The reduction of GH below 2.5 μg/L is commonly accepted as a surrogate endpoint of survival benefit and therefore as a desired therapeutic goal. However, such level of control of disease activity is not obtained in approximately 25%-35% of patients, despite surgery and/or treatment with SSA at full doses. As an attempt to reduce GH level below 2.5 μg/L, doses of 40 mg are sometimes used in the clinical practice. Preliminary evidence indicates that long-term Sandostatin® LAR® 40 mg intramuscular (i.m.) every 28 days was effective in reducing biochemical level of GH and IGF-I, and that the incidence of side effects was scant (Lancranjan et al., 1996).

Pasireotide (cyclo[{4-(NH2-C2H4-NH—CO—O-)Pro}-Phg-DTrp-Lys-Tyr(4-Bzl)-Phe] including diastereoisomers and mixtures thereof—Phg means —HN—CH(C6H5)-CO— and Bzl means benzyl), in free form or in salt form; preferred salts being the lactate, aspartate, benzoate, succinate and pamoate including mono- and di-salts, even more preferred the aspartate di-salt and the pamoate monosalt, most preferred the pamoate monosalt and its synthesis have been described in detail e.g. in WO02/10192, the contents of which are incorporated herein by reference.

In the context of the present invention pasireotide is preferably used as pamoate salt in a long acting dosage form, for instance as microparticles. WO05/046645, the contents of which are incorporated herein by reference, describes that administration of microparticles comprising for instance pasireotide, embedded in a biocompatible pharmacologically acceptable polymer, suspended in a suitable vehicle gives release of all or of substantially all of the active agent over an extended period of time, e.g. several weeks up to 6 months, preferably over at least 4 weeks.

Besides, the GH-receptor antagonist represent a relatively new class of therapy. A currently available agent (pegvisomant, Somavert®) is a genetically engineered GH-receptor antagonist that was developed to compete with naturally occurring GH for binding with the GH receptor.

Unlike native GH, however, this GH-antagonist prevents the dimerization and signaling of GH receptor, resulting in reduced production of IGF-I. In contrast to dopamine antagonists and SSA, GH-antagonist inhibits GH action rather then secretion. Clinical trials have demonstrated that daily subcutaneous administration of pegvisomant monotherapy results in normalization of circulating IGF-I levels in nearly 80 to 90% of patients with acromegaly, with good tolerability. However GH concentrations increased by nearly twofold during therapy, presumably consequent on the fall in IGF-I concentrations (Van der Lely et al., 2001) and whether or not raised GH concentrations is reflected in tumour growth as not yet been answered by clinical studies.

According to a recent publication, treatment of acromegalic patients with the combination of SSA and GH receptor antagonist appears as a feasible option. In an investigator-initiated, 42-week, single centre, open label dose-finding study, 26 acromegalic patients were treated with both a long-acting SSA and weekly administration of the GH-antagonist pegvisomant. Starting dose of pegvisomant was 25 mg per week and was adjusted until serum IGF-I concentration were within the age-adjusted normal range. Monthly treatment with 30 mg of Sandostatin® LAR® or 120 mg of lanreotide Autogel was continued. After 18 weeks, with at least 50 mg of pegvisomant per week, IGF-I concentration was normal in 81% of patients; at week 42, was normal in 95% of patients. The median weekly dose of pegvisomant needed to return IGF-I concentration to normal was 60 mg (range 40-80 mg). Starting from week 12 of administration, mild non-progressive increases in liver transaminases, independent from pegvisomant dose, were recorded in 10 patients (38%). Combined treatment with monthly conventional-dose long-acting SSA and weekly subcutaneous pegvisomant administrations appears promising for medical treatment in acromegalic patients (Feenstra et al., 2005).

In addition, dopamine agonist drugs such as bromocriptine and more recently cabergoline have been employed in acromegalic patients both as single treatment (Abs et al, 1998) and in combination with SSA (Cozzi et al., 2004). In 19 acromegalic patients with active disease and resistant to chronic depot SSA administered at the maximum registered dose, cabergoline was added using the minimal effective and the maximal tolerated dose (range 1-3.5 mg/week). The combined treatment normalized both the biochemical markers (GH<2.5 μg/L and IGF1 for age) in 16% of patients, while the reduction of GH<2.5 μg/L was obtained in 21% and the normalization of IGF1 in 42% patients (Cozzi et al., 2004).

Nevertheless, despite the various treatments available to date, yet a large population of acromegalic patients does not achieve biochemical normalization after at least 6 months of SSA at conventional regimen, i.e. Sandostatin® LAR® (octreotide acetate) at 30 mg i.m. every 28 days, or Autogel® (lanreotide) at 120 mg i.m. every 28 days.

Consequently, given that yet known treatments give insufficiently satisfying results, there is a need for a novel treatment that would permit to overcome the limits and side effects of known treatments, while exhibiting a similar, e.g. at least similar, preferably better, efficacy and safety. In particular, such a new treatment should advantageously permit to successfully treat acromegalic patients not adequately controlled by conventional regimen.

Thus, a first aspect of the present invention concerns a combination containing a long-acting repeatable octreotide acetate, e.g. Sandostatin® LAR® as a first active compound and a second active compound selected in the group consisting of a dopamine-agonist and a growth hormone receptor antagonist, as a combined preparation for simultaneous, separate or sequential use in acromegalic therapy, wherein said long-acting repeatable octreotide acetate is used at 40 mg/28 days, or using pasireotide, preferably pasireotide microparticles as first active compound.

In a preferred embodiment of the present invention, said combined preparation is used for treating acromegalic patients not achieving biochemical normalization after at least six-month treatment using at least one somatostatin analogue at conventional regimen and, in particular, using 40 mg of a long-acting repeatable octreotide, preferably a long-acting repeatable octreotide acetate, every 28 day or pasireotide, preferably pasireotide microparticles.

In the context of the present invention, “biochemical normalization” is meant as mean 1-h GH profile ≦2.5 mcg/L and IGF-1 within the normal ranges, adjusted for age and gender, according to Elmlinger M W et al., Clin. Chem. Lab. Med. 2004, 42(6): 654-664.

For the purpose of the present invention, by “conventional regimen” is meant:
Sandostatin® LAR® at 30 mg i.m. every 28 days or
Autogel at 120 mg i.m. every 28 days.

In the context of the present invention, the acromegalic therapy is preferably administered to the patients during at least 4 months.

In an embodiment of the present invention, the long-acting repeatable octreotide acetate, is injectable. In this respect, the 40-mg dose of said long-acting repeatable octreotide acetate will be conveniently obtained in practice via e.g. two injections of 20 mg each, or one injection of 10 mg and one injection of 30 mg. The injection(s) of long-acting repeatable octreotide acetate are preferably intramuscular, e.g. intragluteal. Alternatively, pasireotide, preferentially pasireotide microparticles can be used accordingly.

On the one hand, according to a particular embodiment, the second active compound used in the product of the invention is a dopamine-agonist and, more particularly, cabergoline.

In this respect, an appropriate dose of cabergoline is from 0.5 mg to 3.5 mg per week. More specifically, the following schema may be advantageously used:

during the first week of therapy: 0.5 mg;

during the second week of therapy: 1.0 mg;

during the third week of therapy: 2.0 mg;

during the fourth week of therapy: 3.5 mg;

during the at least subsequent 3 months of therapy: 1.75 mg or 3.5 mg per week, preferably 3.5 mg per week.

The cabergoline is preferably administered orally, for instance via tablets, e.g. according to the instructions of the manufacturer.

On the other hand, according to another particular embodiment, the second active compound used in the product of the invention is a growth hormone receptor antagonist, preferably pegvisomant.

In this case, an appropriate dose of pegvisomant is 70 mg per week.

Pegvisomant may be advantageously injected, preferably subcutaneously.

A second aspect of the present invention is directed to the use of a long-acting repeatable octreotide acetate at 40 mg/28 days or pasireotide, preferentially pasireotide microparticles, in combination with a second active compound selected in the group consisting of a dopamine-agonist and a growth hormone receptor antagonist, for the preparation of a medicament for treating acromegaly in patients in need thereof.

According to a third aspect, the present invention is related to a method for treating acromegaly in a patient in need thereof, comprising at least administering to said patient:

a) a long-acting repeatable octreotide acetate at 40 mg/28 days, or pasireotide, preferentially pasireotide microparticles, and
b) a second active compound selected in the group consisting of a dopamine-agonist and a growth hormone receptor antagonist.

For both the second and third aspects of the invention, the particular embodiments concerning (i) the patients to be treated, (ii) the long-acting repeatable octreotide acetate, or pasireotide, preferentially pasireotide microparticles, (iii) the second active compound, (iv) the treatment conditions (duration of the therapy, doses of the products, administration routes, etc.), are as defined above.

The term “product” according to the present invention means a combination or a combined preparation or a kit of parts.

The term “package” according to the present invention refers to a unit comprising one or the two active compound(s) together with instructions for administration with the other active compound.

The present invention pertains to the subject matter according to the claims.
The following example part illustrates some embodiments and advantages of the present invention.

### EXAMPLES

The example described hereunder relates to the study of the response to the novel treatment according to the present invention, of patients with biochemically documented acromegaly, not adequately controlled by previous SSA therapy.

The purpose of the study described below is to investigate the efficacy of 8-month treatment of Sandostatin® LAR® monotherapy or Sandostatin® LAR® in combination with either growth hormone (GH) antagonist or dopamine agonist to control both biochemical parameters (GH and IGF-I) in a large population of acromegalic patients that are not adequately controlled after at least 6 months of SSA at conventional regimen.

Previous SSA therapy for acromegaly has been already administered for at least 6 months before inclusion in the study, at conventional regimen, defined as:

Sandostatin® LAR® (octreotide) at 30 mg i.m. every 28 days; or

Autogel® (lanreotide) at 120 mg i.m. every 28 days.

Included patient has:
A measured mean 1-h GH>2.5 μg/L, and
IGF-I above the upper limit, adjusted for age and gender, according to Central Laboratory (Diagn. Lab. Endocrinologie, Kamer Ee 518, Erasmus M C, Dr. Molewaterplein 40, Rotterdam, The Netherlands) range.
   All pre-treatment evaluations are performed within 14 days prior to the 1^{st} drug administration (Day 0).

### STEP 1

Patients are treated for 3 months with Sandostatin® LAR® monotherapy 40 mg i.m. every 28 days (20 mg×2 injections). The day of 1^{st} administration of Sandostatin® LAR® monotherapy 40 mg is Day 0 of the study.

Visit 2 is performed 28 days (+3 days) after the 3^{rd} administration of Sandostatin® LAR® monotherapy 40 mg i.m.

At Visit 2 (end of the 3^{rd} month of treatment) the following biochemical assessments are conducted:
blood sample for IGF-I evaluation is sent to the Central Laboratory in Rotterdam (Erasmus Medical Centre)
three blood samples for GH assessment are sent to the Central Laboratory in Munich. The required schedule of the sampling for the 1-h GH profile is at 0, 30 and 60 minutes.

To allow the transport of blood sample to Central Laboratories, analyses and return of IGF-I and GH values to clinical site, the patient receives a further administration of Sandostatin® LAR® monotherapy 40 mg i.m (20 mg×2 injections).

Visit 3 is performed 28 days (±3 days) after Visit 2.

### STEP 2

As soon as IGF-I and GH values are reported by Central Laboratories, the patient is allocated according to the biochemical response, as follows:
Group 1 (CONTROLLED)
patients with mean GH≦2.5 μg/L and IGF-I within the normal range according to Central Laboratory (adjusted for age and gender) continue to be treated with Sandostatin® LAR® monotherapy 40 mg i.m. every 28 days (20 mg×2 injections) for 4 additional months.
Group 2 (NOT CONTROLLED)
Patients with mean GH>2.5 μg/L and/or IGF-I above the upper limit of normal range according to Central Laboratory (adjusted for age and gender) are randomized by a Interactive Voice Recognition System (IVRS) to be treated as follows:
   Group 2/Arm A—patients add to the previous therapy (i.e., Sandostatin® LAR® monotherapy 40 mg i.m. every 28 days, 20 mg×2 injections) 70 mg s.c. of pegvisomant to be administered weekly.
   This combination therapy is administered for 4 months.
   Group 2/Arm B—patients add to the previous therapy (i.e., Sandostatin® LAR® monotherapy 40 mg i.m. every 28 days, 20 mg×2 injections) oral cabergoline during the evening meal according to the following schema
      1^{st} week >>0.25 mg twice a week (0.5 mg/week)
      2^{nd} week >>0.50 mg twice a week (1 mg/week)
      3^{rd} week >>0.50 mg four time a week (2 mg/week)
      4^{th} week>>0.50 mg daily (3.5 mg/week)
      subsequent 3 months>>0.50 mg daily (3.5 mg/week)

All patients, independently from the ongoing treatment, return for Visit 4 in 2 months. Visit 4 is performed 8 weeks±3 days after Visit 3. During this intermediate visit, liver transaminases, prolactin and fasting blood glucose level together with HbA1c are controlled. Final biochemical assessment are conducted at the End-of-Study Visit (Visit 5: end of 8-months of treatment). Visit 5 is performed 8 weeks±3 days after Visit 4. Patients in Group 1 and Group 2 are classified as ‘Complete Responder’ (CR) if both biochemical parameters are controlled after 8 months of treatment.

A—Treatment

### A.1 Drugs

### a) Sandostatin® LAR®

Sandostatin® LAR®, 40 mg is administered as two injections of 20 mg each, injected into the right and left gluteus regions at the same timeframe, every 28 days.

### aa) Pasireotide

Appropriate dosage of pasireotide may vary. In general, satisfactory results are obtained on administration, e.g. parenteral administration, at dosages on the order of from about 0.2 to about 100 mg, e.g. 0.2 to about 35 mg, preferably from about 3 to about 100 mg of pasireotide per injection per month or about 0.03 to about 1.2 mg, e.g. 0.03 to 0.3 mg per kg body weight per month. Suitable monthly dosages for patients are thus in the order of about 0.3 mg to about 100 mg of pasireotide.

### b) GH-Antagonist (Pegvisomant)

The weekly dose is of 70 mg, administered via subcutaneous injections

### c) Combination of Octreotide+Pegvisomant: Schedule of Treatment

Patient randomized to Group 2/Arm A is administered with Octreotide High Dose 40 mg every 28 days i.m. and subcutaneous injections of pegvisomant at weekly dose of 70 mg, according to the following schedule:

| Step 2 | Sandostatin ® LAR ® 40 mg every | |
|---|---|---|
| Group 2/Arm A | 28 days | Pegvisomant 70 mg/weekly |
| VISIT 3 - Day 0 | 20 mg × 2 i.m. injections | 30 mg + 40 mg s.c. injections |
| Day 7 | — | 30 mg + 40 mg s.c. injections |
| Day 14 | — | 30 mg + 40 mg s.c. injections |
| Day 21 | — | 30 mg + 40 mg s.c. injections |
| Day 28 | 20 mg × 2 i.m. injections | 30 mg + 40 mg s.c. injections |
| Day 35 | — | 30 mg + 40 mg s.c. injections |
| Day 42 | — | 30 mg + 40 mg s.c. injections |
| Day 49 | — | 30 mg + 40 mg s.c. injections |
| VISIT 4 - Day 56 | 20 mg × 2 i.m. injections | 30 mg + 40 mg s.c. injections |
| Day 63 | — | 30 mg + 40 mg s.c. injections |
| Day 70 | — | 30 mg + 40 mg s.c. injections |
| Day 77 | — | 30 mg + 40 mg s.c. injections |
| Day 84 | 20 mg × 2 i.m. injections | 30 mg + 40 mg s.c. injections |
| Day 91 | — | 30 mg + 40 mg s.c. injections |
| Day 98 | — | 30 mg + 40 mg s.c. injections |
| Day 105 | — | 30 mg + 40 mg s.c. injections |

| | |
|---|---|
| VISIT 5 (day 112) - | GH and IGF-I assessment |
| End Of Study | |

### d) Dopamine-Agonist (Cabergoline)

Cabergoline tablets, for oral administration, contain 0.5 mg of cabergoline.

### e) Combination of Octreotide+Cabergoline: Schedule of Treatment

Patient randomized to Group 2/Arm B is administered with Octreotide High Dose 40 mg every 28 days i.m. and oral cabergoline, administered preferably with the evening meal, according to the following schedule:

| Step 2 | | | | | |
|---|---|---|---|---|---|
| Group 2/ | Week | | | Sandostatin ® LAR ® 40 mg | |
| Arm B | TITRATION | | Day | every 28 days | Cabergoline |
| VISIT 3 | \| | 1 | Day 0 | 20 mg × 2 i.m. injections | 0.25 mg twice a week |
| | \| | | Monday and Thursday | | |
| | \| | 2 | Monday and Thursday | — | 0.50 mg twice a week |
| | \| | 3 | Monday-Wednesday- | — | 0.50 mg four times a week |
| | ↓ | | Friday-Sunday | | |
| | | 4 | Day 28 | 20 mg × 2 i.m. injections | 0.50 mg daily |
| | | | Daily | | |
| | | 5 to 7 | Daily | — | 0.50 mg daily |
| VISIT 4 | | 8 | Day 56 | 20 mg × 2 i.m. injections | 0.50 mg daily |
| | | | Daily | | |
| | | 9-11 | Daily | — | 0.50 mg daily |
| | | 12 | Day 84 | 20 mg × 2 i.m. injections | 0.50 mg daily |
| | | | Daily | | |
| | | 13-15 | Daily | — | 0.50 mg daily |

| | | | |
|---|---|---|---|
| VISIT 5 | 16 | Day 112 | GH and IGFI assessment |
| End Of | | | |
| Study | | | |

During the titration period (weeks 1 to 4): adjustments of dose and/or frequency are allowed, providing that within the end of the 4^{th} week the patient is assuming the full dose of cabergoline, namely 3.5 mg/weekly.
During the full-dose period (weeks 5-16): in case of relevant side-effects related to cabergoline and according to medical judgment, the dose is halved to 1.75 mg/week. Once the dose has been halved in a given patient, it is not increased again thereafter.

### A.2 Efficacy Assessments

Efficacy assessments consist in the evaluation of GH and IGF-I serum levels.

B—Data Analysis

### B.1 Variable

The primary efficacy variable is the Complete Response Rate (CRR), defined as the total number of patients who will be “Completely Responder” at the end of 8-months treatment (Visit 5, End-of-Study), whatever is the treatment.

For the purpose of this study, a patient is classified as ‘Completely Responder’ (CR) if both biochemical parameters are controlled at the end of 8-month of treatment, i.e:
GH<2.5 μg/L (according to Central laboratory)

and
IGF-I within the Central Laboratory Normal Range (for age and gender)

The CRR is estimated as the relative number of patients who fulfills the above mentioned definition. The corresponding two-sided 95% CI is calculated for the CRR.

Chi-squared test is applied to compare rates between treatment arms.

### B.2 Secondary Objectives

Secondary efficacy endpoints are:
Complete Response Rate at Visit 2, defined as the total number of patients who are “Completely Responder” at the end of 3-month of treatment with octreotide 40 mg i.m. every 28 days.
Partial Response Rate (PRR) at the End-of-Study Visit, defined as the total number of patients who meet one of the following criteria at the end of 8 months of treatment, whatever was the treatment:
   mean 1 h GH>2.5 mcg/L and <5 mcg/L and either a decrease in IGF-I of at least 50% compared to baseline or IGF-I within normal range
   mean 1 h GH<2.5 mcg/L and a decrease in IGF-I of at least 50% compared to baseline and IGF-I outside normal range

The PRR is estimated as the relative number of patients who fulfills the above mentioned definition. The corresponding two-sided 95% CI is calculated for the PRR.
Improvements of Acromegaly-related clinical signs and symptoms, that are recorded at the baseline and throughout the study. Frequency tables are provided by each visit up to End-of-Study Visit, as well as for changes from baseline.
Health-Related Quality of Life (HRQOL) data, that are collected using the ACROQoL questionnaire and analyzed according to the corresponding algorithm.

The tabulation of laboratory variables (ALT, ALT, fasting glucose, insulin, PRL and HbA1c) and vital signs indicates the normal ranges for each variable. Each value is classified as falling above, below or within normal limits.

C—Notable Laboratory Value Criteria, Special Methods and Scales
IGF-I is measured by a solid-phase, enzyme-labeled chemiluminescent immunometric assay, after sample pretreatment. The assay used is the Immulite-2000 IGF-I, an automated assay system (DPC, Diagnostic Products Corporation, Los Angeles, Calif., USA).
   Reference values for IGF-I, used by the Central Laboratory, are described in Elmlinger et al. (2004).
Analysis of endogenous GH, without interference from pegvisomant is performed by a pegvisomant insensitive assay method.

1. Combination containing (a) Sandostatin® LAR® and (b) a second active compound selected in the group consisting of a dopamine-agonist and a growth hormone receptor antagonist, as a combined preparation for simultaneous, separate or sequential use in acromegalic therapy, wherein said Sandostatin® LAR® is used at 40 mg/28 days.

2. The combination according to claim 1, characterized in that said combined preparation is used for treating acromegalic patients not achieving biochemical normalization after at least six-month treatment using at least one somatostatin analogue at conventional regimen.

3. The combination according to claim 1, characterized in that said conventional regimen is 30 mg of a Sandostatin® LAR® every 28 day.

4. The combination according to claim 1, characterized in that said combined preparation is administered during at least 4 months.

5. The combination according to claim 4, characterized in that the 40-mg dose of said Sandostatin® LAR® is obtained via two injections of 20 mg each.

6. The combination according to claim 5 characterized in that said Sandostatin® LAR® is injected intramuscularly.

7. The combination according to claim 1, characterized in that said second active compound is a dopamine-agonist.

8. The combination according to claim 7, characterized in that said dopamine-agonist is cabergoline.

9. The combination according to claim 8, characterized in that the dose of said cabergoline is from 0.5 mg to 3.5 mg per week.

10. The combination according to claim 9, characterized in that said dose of cabergoline is:
during the first week of therapy: 0.5 mg;
during the second week of therapy: 1.0 mg;
during the third week of therapy: 2.0 mg;
during the fourth week of therapy: 3.5 mg;
during the at least subsequent 3 months of therapy: 1.75 mg or 3.5 mg per week, preferably 3.5 mg per week.

11. The combination according to claim 8, characterized in that said cabergoline is administered orally.

12. The combination according to claim 11, characterized in that said cabergoline is in the form of tablets.

13. The combination according to claim 1, characterized in that said second active compound is a growth hormone receptor antagonist.

14. The combination according to claim 13, characterized in that said growth hormone receptor antagonist is pegvisomant.

15. The combination according to claim 14, characterized in that said pegvisomant is administered at 70 mg per week.

16. The combination according to claim 14, characterized in that said pegvisomant is injectable.

17. The combination according to claim 16, characterized in that said pegvisomant is injected subcutaneously.

18. Combination containing (a) Pasireotide and (b) a second active compound selected in the group consisting of a dopamine-agonist and a growth hormone receptor antagonist, as a combined preparation for simultaneous, separate or sequential use in acromegalic therapy.

19. The combination according to claim 18, characterized in that said dopamine-agonist is cabergoline.

20. The combination according to claim 18, characterized in that said growth hormone receptor antagonist is pegvisomant.

21. A package comprising the combination according to claim 1 together with instructions for administration to a patient in need thereof.

22. A method of treating acromegaly wherein the combination according to claim 1 is administered to a patient in need thereof.

23. (canceled)
